(19) United States
(12) Patent Application Publication
(10) Pub. No.: US 2024/0050615 A1
(43) Pub. Date: Feb. 15, 2024

(54) Wifi LED, audio, jewelry, toy lamp, fragrant diffuser

(71) Applicant: ANDREW WALKER, JR., Culver City, CA (US)

(72) Inventor: ANDREW WALKER, JR., Culver City, CA (US)

(21) Appl. No.: 18/142,945

(22) Filed: May 3, 2023

(51) Int. Cl. A61L 9/03

(52) U.S. Cl. CPC A61L 9/03; A61L 2209/135

(57) ABSTRACT

The decorative jewelry lamp includes an apparatus of audio, jewelry, Wi-Fi, an LED light source, an entertainment toy, an infusion of fragrantly volatile molecule agents, a curvy design that uniquely volatile aroma out of the lamp, and an LED smart bulb controlled by Wi-Fi, encrusted with precious gems, gold, silver and platinum metal straps on the outer layer of the apparatus, and an LED light source, sound for entertainment for kids, and sleep therapy in conjunction with working parts inside of the apparatus. By removing all the inner parts of the lamp, it becomes a nonfunctional decorative jewelry accessory or storage for kid's toys. All prior art, such as jewelry, fragrant lamps, LED light sources, and toys, is void of this invention's aspects and multifunctional elements.

### BACKGROUND

### Field of the Invention

Embodiments disclosed herein generally relate to Wi-Fi, LED, jewelry, toys, lamps, indoor ornaments such as jewelry, and audio aroma diffusers.

### Description of the Related Art

Owning a piece of jewelry has brought timeless joy to the individual. With aroma therapy on the rise, this invention has a new method of combining aromas, audio, and lighting entertainment, all in a one-piece apparatus.

For instance, a Warmers for scented oils bearing US patent 2,011,0286726A1 is issued to Party lite Worldwide Inc. The patent discusses a warmer for the scented oil is provided. The warmer includes a base, a removable cover position on the base, and a warming device. The cover has a substantially spherical shape with an opening at an upper apex and an open channel formed in the cover at the upper apex around an outer periphery of the opening. The open channel is configured to receive the scented oil. The warming device is in a heat-conductive relationship with the cover so that the warming device warms scented oil in the end channel. The warming device can be one or more lighting devices with an incandescent light bulb, a resistance heater with or without a lighting device such as a light-emitting diode, or a candle.

Another patent on a Candle-powered aroma generator bearing U.S. Pat. No. 4,781,895A is issued to Donald Spector. The patent discusses a candle-powered aroma generator in a lantern format housing a candle holder within a cylindrical container with a translucent mounted on a base and a removable cover whose roof has a central well. Below the bottom of the well is the burning wick of a candle supported in the holder, the candle flame heating the bottom, which then functions as a miniature hot plate. Received in the well and sting on the hot plate is a replaceable aroma cartridge within whose cavity is a porous pad that partitions the cavity into upper and lower air chambers. The lower chamber is in heat transfer in relation to the hot plate, and the upper chamber has a vent. The pad is impregnated with a volatile liquid fragrance, and the heated air in the lower chamber produces a positive pressure therein, which forces the air through the pad to volatilize the fragrance. The resultant vapor is discharged into the atmosphere through the vent.

Another patent on a Scent-generating lamp using mating parts bearing U.S. Pat. No. 4,965,490A is issued to Osram Sylvania Inc. The patent relates to a vapor-generating electric lamp. It includes a sealed lamp envelope having a concave depression therein, an incandescent filament mounted within the lamp envelope, a lamp base, and a replaceable, solid insert in the concave depression. The insert produces a scent when warmed by heat from the filament. Preferably, the insert is a polyamide impregnated with a scent and includes a portion that matches the shape of the depression in the lamp envelope. The depression for holding the insert is preferably located at the opposite end of the lamp from the base. When the lamp is used in a base-down orientation, the insert is held in the depression by gravity.

Another patent on an Aroma dispenser for candle bearing U.S. Pat. No. 6,328,935B1 is issued to Custom Essence Inc. The patent relates to an aroma dispenser comprised of a flame source for providing heat, an open, porous annular ceramic substrate adapted for absorbing and reversibly retaining a quantity of at least one aromatic fluid therein, a support structure for holding the porous substrate in a coaxial spaced-apart relationship above the flame source, whereby heat generated by the flame source induces the retained aromatic fluid within the substrate to disperse into the ambient air.

A patent on warmers for scented oils bearing US patent 2,011,0286726A1 is issued to Partylite Worldwide Inc. The patent relates to a warmer for scented oil is provided. The warmer includes a base, a cover removably positioned on the base, and a warming device. The cover has a substantially spherical shape with an opening at an upper apex and an open channel formed in the cover at the upper apex around an outer periphery of the opening. The open channel is configured to receive the scented oil. The warming device is in a heat conductive relationship with the cover so that the warming device warms scented oil in the open channel. The warming device can be one or more lighting devices with an incandescent light bulb, a resistance heater with or without a lighting device such as a light-emitting diode, or a candle.

Another Canadian patent, 2,449,734A1 on Candle Warner, is issued to Pende Inc. The patent relates to a scent dispersion system (10), including a heating device (14) having a top surface and a container (16) of scented wax disposed upon the top surface of the heating device (14). Upon heating, the scent from the wax is caused to disperse into the surrounding environment. The system (10) may further include an air-circulating fan (34) associated with the heating device (14) for circulating air around the container (16) of scented wax to help disperse the scent.

Multiple inventions have been proposed to advance and enhance utility regarding warmers for scented oils and wax fragrances. However, it has been determined by the present disclosure that there is a need for warmers for scented oils that overcome, alleviate, and mitigate one or more deleterious effects of prior art warmers.

None of the previous inventions and patents, taken either singly or in combination, is seen to describe the instant invention as claimed. Hence, the inventor of the present invention proposes to resolve and surmount existent technical difficulties to eliminate the shortcomings above of prior art.

A patent on a jewelry accessory bearing US patent US20050034480A1 is issued to Christopher Martz. A fragrant decorative jewelry accessory includes an infusion of fragrantly volatile molecules and an adhesive on the underside of the accessory intended to attach the accessory removably. Fragrance may be infused on the accessory by soaking an absorbent material with the fragrantly volatile molecules through a printing technique, attaching encapsulated fragrance molecules onto the accessory, or attaching a fragrance layer onto the accessory. The fragrant decorative accessory can be removably attached to clothing, a bra strap, a choker, or a hair accessory.

Another US patent, US20150310625A1, is issued to David SAATHoff; this device is another jewelry fragrant emitting apparatus, Devices, and methods for long-term sustained release of a fragrant compound. Exemplary embodiments may comprise a channeled member with nanochannels or microchannels to sustain the fragrant compound's release.

Another Japanese patent, JP5443671B2 by [name], is another fragrant jewelry emitting apparatus. The present invention also relates to fragrant trinkets, jewelry, or ornaments. More specifically, the present invention relates to indoor ornaments such as jewelry (jewelry), jewelry, ornaments, daily necessities, and figurines.

Another Chinese patent, CN205494974U, by [nag]. The utility model belongs to the technical field of the toy, specifically providing lasting fragrant jewelry, which comprises a housing equipped with a fragrant package in the casing. A fragrant package comprises a gas permeability sealing bag and a plurality of fragrant particles held in the sealing bag, the bottom of the sealing bag is fixed in the casing, and the side reason of the sealing bag is equipped with easily tears the mouth and it has the tractive line easily to tear open-ended upside rigid coupling, and the other end of the tractive line passes the through-hole at casing top, and the tractive line rigid coupling that stretches out the through-hole outer has the pull ring. The utility model provides lasting fragrant jewelry; inside, it is equipped with the fragrant package, puts, transports, and sells the process into production, and the fragrant smell in the fragrant package cannot be distributed away. When treating the consumer, the perfume package that pulls the tractive line will seal up when opened, and a fragrant smell is distributed in the fragrant package. This jewelry can keep a fragrant smell reaching the consumer's hand for a long time.

Another Japanese patent, JP2004216106A by Taiji Enguchi, provides a fragrant ornament that releases fragrance only when people who use the ornament change the shape of the ornament by pulling, kneading, and squashing. <P>SOLUTION: The fragrant ornament is equipped with a fragrance container comprising elastic material, so a portion can internally house a fragrance. The fragrance container has fragrance-release pores that completely or approximately hold the aromatic substance inside and generally release fragrance when the shape of the fragrance container is changed.

Another Chinese patent, CN107631732B, was issued to Zhejiang Deqing Zhilu Navigation Research Institute Co., Ltd. The invention belongs to the field of indoor positioning. It discloses a mixed indoor positioning method and a system based on Bluetooth, Wi-Fi, and sound waves, wherein a base station is used for broadcasting a Wi-Fi signal, a Bluetooth signal, and an audio signal subjected to time alignment and signal modulation; the user side receives the RSSI of the Bluetooth and Wi-Fi signals, demodulates the transmitting time of the audio signal and the broadcast message of the Bluetooth signal, and realizes the mixed ranging position calculation based on the TOA and the RSSI; the positioning system includes a mixing chamber positioning base station and user equipment; the ranging links are realized between base stations and between the base stations and the user terminal of the system. The invention has stable and continuous positioning results and a small influence on signal shielding. Based on TOA, audio signal range finding can avoid the jump point that RSSI is unstable and the signal shelters from and causes, uses the mode of range finding and speed measuring to guarantee that the positioning result is continuously reliable simultaneously.

Another US patent, US20100102142A1, is issued to Daria Tagliareni. Scent dispenser assemblies for an extended time-release of a desired scent are disclosed herein. In some embodiments, a scent dispenser assembly may include a substrate configured to release a desired scent over time and a housing for retaining the substrate configured to be worn by a user. In some embodiments, a scent dispensing system may include a plurality of substrates, each configured to release a desired scent over some time and having a three-dimensional shape, wherein the three-dimensional shape is at least one of a sphere or a polyhedron and housing for retaining the plurality of scent dispensers.

Another South Korean patent, KR102324436381, is issued to [name]. The present invention provides an audio-sharing method and system that aims to revolutionize how people listen and share music and to bring multiple uses to a wireless headphone referred to as a HEDphone. A communication protocol, called the HEDtech protocol in HED systems, allows users to share music between multiple HED phones while using a single audio source. A wireless connection is established between the HED phone and the mobile device containing the audio source while having the ability to allow other HED phone users to join wirelessly and listen to the same audio source. A feature of Super Human Hearing (SHH) is that it overcomes the existing ANR (ambient noise reduction) jumpover.

### SUMMARY

The present invention also relates to a fragrant decorative lamp, trinkets, toys, jewelry, or ornaments; more specifically, the present invention relates to lamps with audio, toys, indoor ornaments such as jewelry, jewelry ornaments, daily necessities, figurines, Wi-Fi, and aroma diffusers.

The present invention's general object is to provide an improved composition of a lamp for relaxing, wearing as jewelry ornaments, improving sleep, and providing entertainment and aroma therapy.

This includes a one-piece apparatus in a curvy genie bottle shape that gives the current flow of aroma agents from the bottom of the apparatus to the top, which directs the aroma to be released out of the crown's apertures in conjunction with inner working parts, with a base portion that has an aperture in which the LED light can pass through, all inner parts can be removed from the lamp so that the user can insert any toys inside of the lamp or could be worn as jewelry, in this manner the apparatus is nonfunctional. The cover is substantially curvy with a crown cone-shaped top piece, or the crown can be manufactured in any particular shape. The lamp can be an aroma therapy unit in which the end user can insert any type of aroma agents into the base of the lamp inside of the bowl to get the desired effect or way of the resistance heater rod or lighting devices located at the base of the apparatus, the smart bulb signals and audio signals the user side receives or sends demodulates the audio signal and the broadcast text of the Bluetooth signal, transmitting the signals to the LED smart light bulb. The lamp is in a heat-conductive relationship with the aluminum alloy container or the LED smart light bulb so that the aroma agents can be volatile at the desired effect. The lamp can be a resistance heater rod or a lighting device with a smart LED bulb developed with polychrome technology. This gives the user Wi-Fi capabilities to turn the apparatus on and off, select the color of the smart light bulb temperature, and control the lamp remotely. Also, the apparatus has a fan to control heat and temperature to protect the LED smart bulb. The apparatus also provides a speaker for sound connected to the grille so that sound may travel through the grille of the base of the apparatus. The apparatus dimensions can be manufactured depending on the desired effect.

The apparatus LED smart bulb has a set of light-emitting elements to emit visible light having a fixed color parameter and another set of light-emitting elements configured to emit visible light having another color parameter different from the first color parameter wherein the target color parameter value is received from the communication module.

The apparatus can be, in some embodiments, an entertainment device, a piece of jewelry to be worn, a toy, or a lighting device with a smart LED light bulb with polychrome technology utilizing over six (6) million colors. The apparatus can be secured to a base of the enclosure so that the enclosure shields substantially all of the LED smart light bulb and all other working parts. Here, the enclosure can include a decorative pattern encrusted with gems, gold, and silver strappings defined on an external surface, and one or more portions of the enclosure can be at least partially translucent. In other embodiments, the lamp can also be a toy for aroma therapy, sleep therapy, and entertainment while the LED smart bulb is inserted. The lamp can also be, in other embodiments, a resistance heater rod. The resistance heater rod can be secured to the base of the enclosure. In these embodiments, the lamp can optionally include a lighting device such as, but not limited to, a light-emitting diode. The apparatus is still other embodiments can be a heating steam rod or LED smart light bulb that is removable position able within the enclosure so that the enclosure shields substantially all of the heating rod or LED smart bulb with the heating rod or LED smart light bulb being at a center of the base opening and centered below the aroma bowl.

The apparatus can optionally include at least one vent opening defined in the enclosure. For example, the vent opening can be a plurality of evenly spaced openings near the bottom of the enclosure for the introduction of the aroma bowl container and ventilation for the LED smart light bulb. A lamp for releasing aroma agents, sleep therapy, and jewelry to be worn, having a base, crown cover, with vent openings defined between the crown and the base, heating rod, and LED smart light bulb that is provided. The aroma bowl is centered inside the base of the lamp to receive the scented aroma agents therein to be viol tiled.

The heating rod or the LED smart light bulb is removable in the base, with the heating rod or the LED smart light bulb located at the center of the lamp's base. In this manner, the heating channel container is in a heat conductive relationship with the heating rod or the LED smart light bulb so that aroma agents in the aroma bowl can be warmed and volatile by either the heating rod, LED smart light bulb, or the self-emitting aroma agents.

The apparatus's fan assembly is supported on the base of the lamp, a housing having an air inlet, an air outlet, a motor, and a fan driven by the motor; the fan is supported in the interior of the housing and is operable to bring airflow into the lamp for temperature control.

The apparatus's speaker assembly is supported on the base of the lamp, a grille connected to the side/housing and is connected and supported directly to the speaker; sound may travel through the grille in which the fan, LED smart light bulb, speaker, the heating rod may be placed in various ways attached to the base.

In various embodiments, the crown cover can include a decorative pattern defined on an external surface, and the crown cover can be at least partially translucent.

### BRIEF DESCRIPTION OF DRAWINGS

FIG. 1 is an exploded perspective view of a Wi-Fi, LED, Audio, Jewelry, Toy lamp, and Fragrant diffuser, showing my new multifunctional lamp and illustrating a one-piece genie bottle-shaped lamp with a top crown portion that is connected to the base according to an exemplary embodiment of the present disclosure.

FIG. 2 is a rear perspective view of the lamp of FIG. 1, having portions of the cover cut away to show the first and second embodiment of the lamp according to an exemplary embodiment of the present disclosure.

FIG. 3 is a second side view of the lamp of FIG. 1, offset from the first side view of FIG. 1 by one hundred and eighty degrees.

FIG. 4 is a right-side elevational view of the invention.

FIG. 5 is a top-plan view of the invention.

FIG. 6 is a bottom plan view of the invention, and,

FIG. 7 is a front perspective view of the lamp of FIG. 1, having portions of the cover cut away to show the embodiments of a multifunctional lamp, two of the embodiments of the warming devices (e.g., LED, rod), fan, air vent, and speaker according to an exemplary embodiment of present disclosure.

Skilled artisans will appreciate that elements in the figures are illustrated for simplicity and clarity and have not necessarily been drawn to scale. For example, the dimensions of some of the elements in the figures may be exaggerated relative to other elements to help improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION OF THE INVENTION

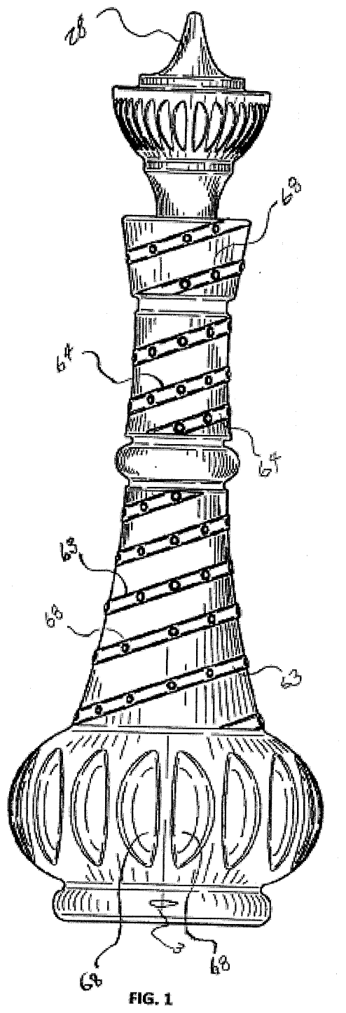

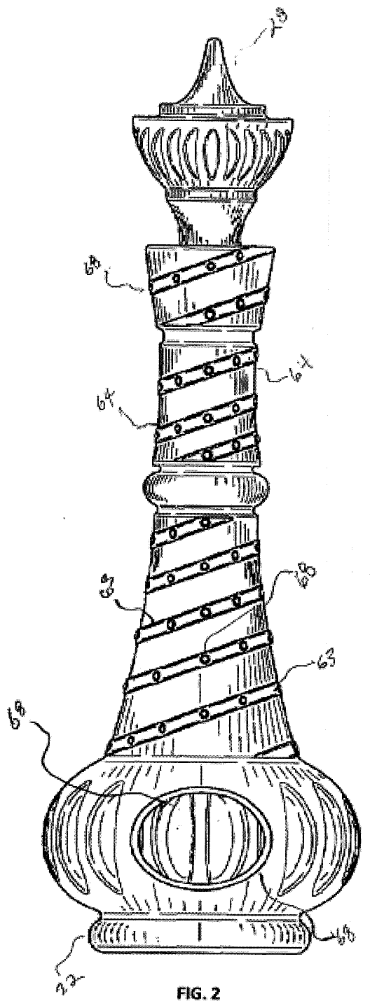

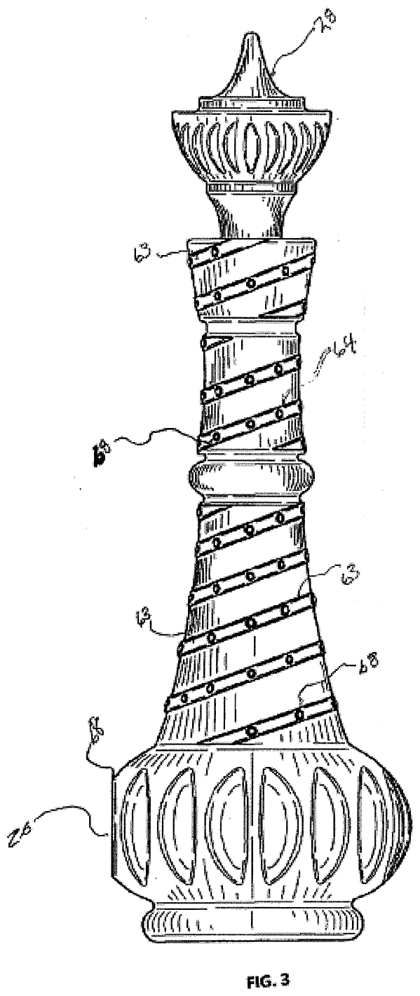

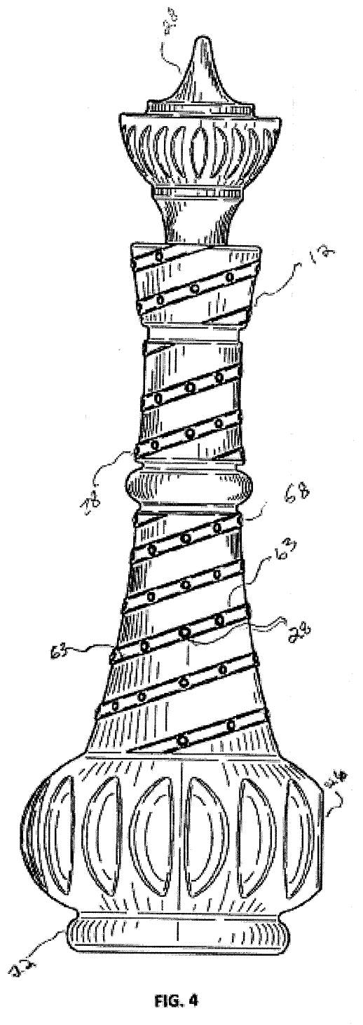

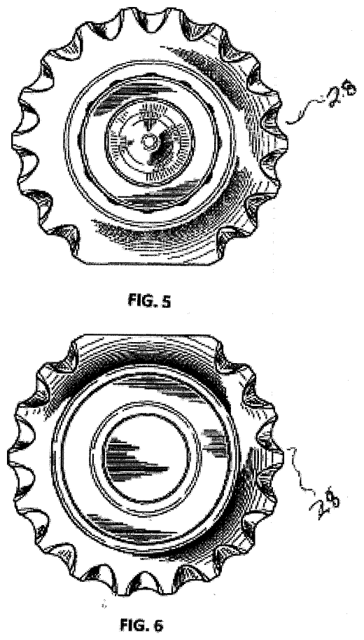

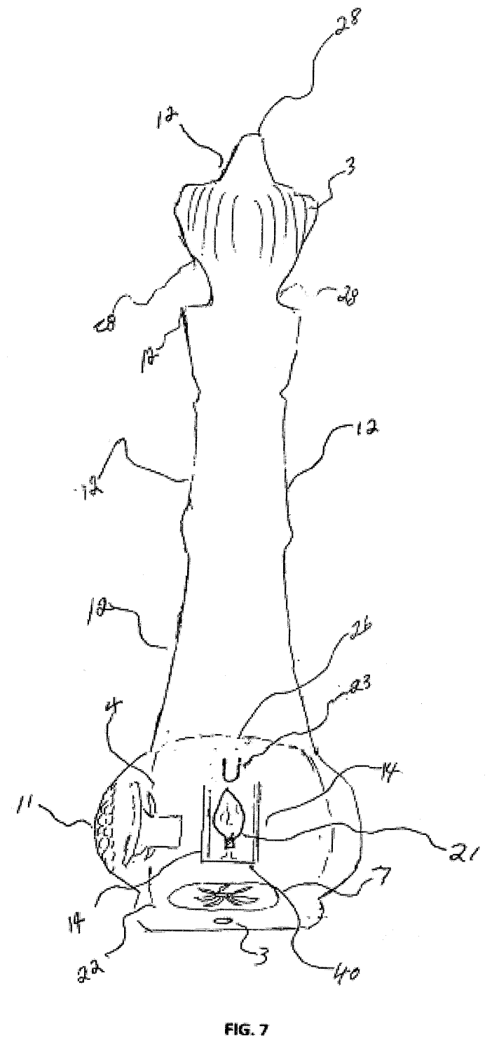

Referring to the drawings and FIG. 1 through 7, an exemplary embodiment of a lamp for Wi-Fi, LED, Audio, Jewelry, Toy lamp, and Fragrant diffuser is shown. It is generally referred to by reference number 12. Lamp 12 includes the curvy design (included in the drawings) to give the unique aroma effect to be released and an enclosure 12 and warming devices 21 and 14. In the embodiment of FIG. 7, warming device 21 is illustrated by example as an LED smart light bulb 21, shown in phantom. The resistance heating rod 14 is illustrated by an aluminum pipe connected to the lamp's aroma bowl 23 and fan 7 for current control and speaker 4, which outputs audio sound from the said lamp.

Advantageously, lamp 12 is configured to use the heat generated by warming device 21 or the heating rod 14 to warm scented aroma agents (not shown) within an aroma bowl 23 defined in the enclosure, where the open aroma bowl 23 is configured to receive scented aroma agents therein, in the embodiment of FIG. 7. As used herein, the term “aroma agents” shall mean any composition, in a liquid form, a semi-solid form, or a solid form, essential oils, that includes a scent within the composition. In contrast, the scent is released upon heating the composition to a desired temperature at a known release rate.

In the illustrated embodiment, enclosure 12 includes components: one piece with a crown cover 28, a base 22, and an aroma bowl reservoir 23. Of course, it is contemplated by the present disclosure for enclosure 12 to be a single unitary component or for the enclosure to include more than three components, which fit together to form the desired enclosure around the lamp device 12 wherein said least one vent 3 openings comprise a plurality of evenly spaced indentations defined in said cover 12.

Cover crown 28 includes apertures to release the aroma into the atmosphere that sits on the top of the lamp 12 and as 26 defined therethrough. Open container aroma bowl 6 is formed in the base of lamp 12 above the heating rod 14, and LED smart light bulb 21 Opening 26 allows light and heat generated by the bulb 21 or heating rod 14 to volatile the aroma agent. In addition, cover 12 is in a heat conductive relationship with warming devices 21 and 14 so that the aroma agents in the open container 23 are warmed by ether warming device 21/14 or (self-emitting aroma agents), and the curvy shape allows the aroma agent's currents to flow upwards out of the cover 12.

Wi-Fi-enabled smart light bulbs 21 and rod 14 are repositioned on base 22 to cover 12 shields or substantially enclose all of the smart bulb 21 and rod 14 so that the smart bulb 21 and rod 14 are at the center of opening 26. In this manner, lamp 12 allows light from the smart light bulb 21 or heat from rod 14 to exit through the top of the cover 12 of the container reservoir aroma bowl 23, yet still provides sufficient heat to release the scent from the scented aroma agents located inside of the container reservoir 6. Also, the crown cover 28 has apertures located within the cover of it; this way, the user can release the aroma through the crown 28.

In other embodiments, cover 12 can include one or more decorative patterns 36 defined on external surface 12. lamp 12 can be powered on and off with the push button 33 on the base of the lamp 12 or with Wi-Fi through the smart bulb 21. Also, it is contemplated by the present disclosure for cover 12 to be at least partially translucent to allow at least a portion of the light generated by the LED smart light bulb 21 to escape cover 12. As used herein, the term ‘translucent’ shall mean that at least some visible light can pass through cover 12 by way of openings in cover 12 and the property of the materials from which cover 12 is made, allowing light to pass through.

Enclosure 12 can be formed of any material conducting heat from warming device 21 or rod 14, open container reservoir 23, and provide, when desired, the aforementioned translucent property and LED lighting desired by the LED smart bulb in a preferred embodiment. Enclosure 12 comprises a material selected from the group: glass, ceramic, plastic, gold 63, silver 64, gems 68, and any combinations thereof. Cover 12 is shown as having a substantially curvy genie bottle shape in the illustrated embodiments. Of course, it is contemplated the present disclosure for cover 12 to have any desired shape. Gems, 68, gold strapping 63, and silver or platinum strapping 64 on the exterior of the cover 12.

Referring to FIG. 7, lamp 12 is illustrated using an LED smart light bulb 21 as a controlling device controlling the speaker 4 and fan 7 and as a warming device 21. Here, the smart bulb 21 and the rod 14 are formed as part of a lighting device 40 secured to base 22. Cover 12 shields substantially all of rod 14 and LED smart bulb 21. As discussed in detail above, cover 12 can include a decorative pattern 36 defined on an external surface 12 thereof, and the cover can be at least partially translucent. Lighting/heating device 21/14 can be selectively placed in electrical communication with a power source (not shown), such as, but not limited to, a battery or an electrical outlet. Some said base 22 vent openings 3 are defined between cover 12 and base 22. Fan 7 and vent opening 3 can ensure that the LED bulb 21 has sufficient oxygen and cool air to operate properly and provides a convective air current through lamp 12. Enclosed in a separate compartment are fan 7 and LED smart bulb 21 at the base 22 of the cover 12. The LED smart bulb 21 can stand alone for entertainment and sleep therapy toys for kids and provide a center inside the cover 12 compartment to store their small toys at the base 22 of the lamp. Also, 12 in smaller dimensions can be worn as aroma therapy jewelry when the user inserts aroma agents into the aroma bowl 23 at the base 22 of the lamp 12.

1. A Wi-Fi, LED, Audio. Jewelry, Toy lamp, Fragrant diffuser comprising of Wi-Fi, LED, audio sound waves, jewelry, toy lamp, and fragrant diffuser that infuse fragrantly volatile molecules outside of the lamp, a curvy design which operates the aroma current flow; and
A Wi-Fi, LED, Audio. Jewelry, Toy lamp, Fragrant diffuser in claim 1 whereas the lamp is any metal material,
A Wi-Fi, LED, Audio. Jewelry, Toy lamp, Fragrant diffuser lamp as in claim 1 whereas it is plastic,
A Wi-Fi, LED, Audio. Jewelry, Toy lamp, Fragrant diffuser claim 1, whereas the lamp infuses fragrance by inserting the aroma agents into the aroma bowl with fragrantly volatile molecules.
A Wi-Fi, LED, Audio. Jewelry, Toy lamps, and Fragrant diffusers claim 1, whereas fragrance is volatile through the control of Wi-Fi, smart light bulbs,
A Wi-Fi, LED, Audio. Jewelry, Toy lamps, and Fragrant diffusers, as in claim 1, whereas fragrance is volatile through the heating rod heating source,
A Wi-Fi, LED, Audio. Jewelry, Toy lamps, and Fragrant diffusers, as in claim 1, whereas fragrance can be volatile while wearing the lamp as jewelry by the self-emitting aroma agents,
A Wi-Fi, LED, Audio. Jewelry, Toy lamp, Fragrant diffuser as in claim 1, whereas toys can be stored inside of the lamp (storing or removing toys gives the apparatus no functions),
A Wi-Fi, LED, Audio. Jewelry, Toy lamps, and Fragrant diffusers claim 1, whereas the audio sound can be activated through Wi-Fi-operated speakers,
A Wi-Fi, LED, Audio. Jewelry, Toy lamp, Fragrant diffuser as in claim 1 whereas the fan can keep the LED smart bulb cool while inside the lamp whereas is Wi-Fi controlled,
A Wi-Fi, LED, Audio. Jewelry, Toy lamp, Fragrant diffuser as in claim 1, whereas the lamp is an entertainment toy,
A Wi-Fi, LED, Audio. Jewelry, Toy lamp, and Fragrant diffuser as in claim 1, whereas the lamp's inner parts can be removable and replaced with toys (which gives the apparatus no functions.)
A Wi-Fi, LED, Audio. Jewelry, Toy lamp, and Fragrant diffuser as in claim 1, whereas the aroma bowl can be inserted in the lamp,
A Wi-Fi, LED, Audio. Jewelry, Toy lamps, Fragrant diffuser, as in claim 1, whereas the aroma bowl can be filled with aroma agents,
A Wi-Fi, LED, Audio. Jewelry, Toy lamp, Fragrant diffuser as in claim 1, whereas the lamp is an aroma therapy volatile unit,
A Wi-Fi, LED, Audio. Jewelry, Toy lamp, Fragrant diffuser as in claim 1, whereas the lamp is a plastic toy (minis inner parts),
A Wi-Fi, LED, Audio. Jewelry, Toy lamp, Fragrant diffuser as in claim 1, whereas the lamp is a piece of jewelry (minis inner parts),
A Wi-Fi, LED, Audio. Jewelry, Toy lamp, Fragrant diffuser as in claim 1 whereas the lamp is shaped as a curvy genie bottle shape to allow the aroma agents to flow upwards and out of the top of the lamp in a unique fashion in which the design is part of the whole operating function of the apparatus,
A Wi-Fi, LED, Audio. Jewelry, Toy lamp, Fragrant diffuser as in claim 1, whereas the lamp is made out of gold,
A Wi-Fi, LED, Audio. Jewelry, Toy lamp, Fragrant diffuser as in claim 1, whereas the lamp is made out of silver,
A Wi-Fi, LED, Audio. Jewelry, Toy lamps, and Fragrant diffusers comprise decorative gold, silver, and platinum metal straps with diamonds, rubies, and emeralds infused with a fragrance designed to be worn as a ring, necklace, or earring jewelry.
